(19) United States
(12) Patent Application Publication
(10) Pub. No.: US 2026/0000333 A1
(43) Pub. Date: Jan. 1, 2026

(54) SYSTEM FOR MONITORING HEART CONDITION FOR ANIMAL

(71) Applicant: CARESIX INC., Jeju-si (KR)

(72) Inventors: Sung Hoon KIM, Jeju-si (KR); Jin Wook CHANG, Jeju-si (KR)

(73) Assignee: CARESIX INC., Jeju-si (KR)

(21) Appl. No.: 19/251,722

(22) Filed: Jun. 26, 2025

(30) Foreign Application Priority Data
Jun. 28, 2024 (KR) 10-2024-0085131
Jun. 13, 2025 (KR) 10-2025-0077824

(51) Int. Cl. A61B 5/282; A61B 5/00; A61B 5/11; A61B 5/256; A61B 5/277; A61B 7/02

(52) U.S. Cl. CPC A61B 5/282; A61B 5/1102; A61B 5/256; A61B 5/277; A61B 5/6802; A61B 5/7267; A61B 7/02; A61B 2503/40; A61B 2562/0219

(57) ABSTRACT

Disclosed is a system for monitoring a heart condition in a companion animal. The system can collect electrocardiogram data of a companion animal through a capacitive electrocardiogram sensor without prior preparation such as hair removal, collect phonocardiogram data and ballistocardiogram data through a phonocardiogram sensor and a ballistocardiogram sensor, and generate cardiogram-integrated data available for diagnosing a heart condition in the companion animal by merging the electrocardiogram data, the phonocardiogram data, and the ballistocardiogram data.

### CROSS-REFERENCE TO RELATED APPLICATIONS

This application claims the benefit under 35 USC § 119(a) of Korean Patent Application No. 10-2024-0085131, filed on Jun. 28, 2024 and Korean Patent Application No. 10-2025-0077824, filed on Jun. 13, 2025, in the Korean Intellectual Property Office, the entire disclosure of which is incorporated herein by reference for all purposes.

### BACKGROUND

### 1. Technical Field

The present disclosure relates to a system for monitoring a heart condition in a companion animal, and more particularly, to a system for monitoring a heart condition by integrating the cardiograms of a companion animal through a non-restrained wearable device that integrates electrocardiogram, ballistocardiogram, and phonocardiogram generated according to the heart motion of the companion animal.

### 2. Related Art

Recently, as the number of households raising companion animals increases, companion animal diseases are also increasing. The increase in the companion animal diseases leads to economic and emotional burdens on owners, but it is not easy to early recognize and treat the diseases. Most companion animal diseases are detected through close observation by the owner, but when symptoms are mild or absent, it is very difficult for owners without specialized knowledge of the diseases to detect the diseases. In particular, in the case of a heart condition, it is difficult to ascertain symptoms at the onset of the disease. Therefore, after the condition worsens to a serious degree, the owner visits an animal hospital, resulting in a high mortality rate. Accordingly, it is very important to early diagnose and manage an asymptomatic heart condition through regular heart checkups.

As disclosed in the Patent Document of the following prior art Document, an electrocardiogram (ECG) is measured to diagnose a heart condition in a companion animal in the related art. The electrocardiogram is an analysis of the electrical activity of the heart in the form of waves and can be measured by bringing electrodes into contact with the skin of the companion animal. However, according to the electrocardiogram measurement method in the related art, since the electrodes need to be brought into contact with the skin of the companion animal, prior preparation such as hair removal for exposing the skin is required. In addition, since the electrodes may fall off the skin due to the movement of the companion animal, the companion animal needs to be fixed so that the movement is limited. Otherwise, it is difficult to accurately measure the electrocardiogram.

Therefore, a solution to solve the problems of the technique for monitoring a heart condition in a companion animal in the related art is urgently required.
Patent document 1: KR 10-2016-0014282 A

### SUMMARY

The present disclosure is intended to solve the problems of the above-described related art, and one aspect of the present disclosure is to provide a system for monitoring a heart condition in a companion animal that collects electrocardiogram data of a companion animal through a capacitive electrocardiogram sensor without prior preparation such as hair removal, collects phonocardiogram data and ballistocardiogram data through a phonocardiogram sensor and a ballistocardiogram sensor, and generates cardiogram-integrated data available for diagnosing a heart condition in the companion animal by merging the electrocardiogram data, the phonocardiogram data, and the ballistocardiogram data.

A system for monitoring a heart condition in a companion animal according to an embodiment of the present disclosure includes: a capacitive electrocardiogram sensor including a first electrode, a second electrode, and a third electrode not in contact with a skin of a companion animal and collecting an electrocardiogram signal of the companion animal based on capacitive coupling; a phonocardiogram sensor that collects a phonocardiogram signal of the companion animal; a ballistocardiogram sensor that collects a ballistocardiogram signal of the companion animal; a sensor-integrated body in which the capacitive electrocardiogram sensor, the phonocardiogram sensor, and the ballistocardiogram sensor are arranged; a wearable member that is attached with the sensor-integrated body and is worn by the companion animal, the sensor-integrated body being arranged toward a heart area of the companion animal; a datafication unit that generates electrocardiogram waveform data, phonocardiogram waveform data, and ballistocardiogram waveform data by datafying the collected electrocardiogram signal, phonocardiogram signal, and ballistocardiogram signal over time; a data storage unit that stores the generated electrocardiogram waveform data, phonocardiogram waveform data, and ballistocardiogram waveform data; a data merging unit that generates cardiogram-integrated data by merging the generated electrocardiogram waveform data, phonocardiogram waveform data, and ballistocardiogram waveform data; and a display that displays the cardiogram-integrated data.

In the system for monitoring the heart condition in the companion animal according to an embodiment of the present disclosure, in the sensor-integrated body, the phonocardiogram sensor may be arranged at a center of the sensor-integrated body, one first electrode, one second electrode, and one third electrode may be arranged at vertices of an imaginary triangle centered on the phonocardiogram sensor, respectively, and the ballistocardiogram sensor may be arranged between two of the first electrode, the second electrode, and the third electrode.

In the system for monitoring the heart condition in the companion animal according to an embodiment of the present disclosure, one surface of the sensor-integrated body that faces a skin of the companion animal may be provided with an internal space in which the phonocardiogram sensor is embedded and a through hole through which heart sounds are introduced into the internal space.

In the system for monitoring the heart condition in the companion animal according to an embodiment of the present disclosure, the first electrode may be arranged between two front legs of the companion animal.

The system for monitoring the heart condition in the companion animal according to an embodiment of the present disclosure may further include: an acceleration sensor mounted on the sensor-integrated body and measuring acceleration; a motion determination unit that digitizes a movement of the companion animal based on the acceleration measured by the acceleration sensor; an abnormal signal classification unit that classifies, as an abnormal signal, the electrocardiogram signal measured in an abnormal signal time interval in which a movement value digitized by the motion determination unit exceeds a predetermined threshold value; and an electrocardiogram signal correction unit that corrects the electrocardiogram waveform data in the abnormal signal time interval.

In the system for monitoring the heart condition in the companion animal according to an embodiment of the present disclosure, based on the electrocardiogram waveform data, the phonocardiogram waveform data, and the ballistocardiogram waveform data that are measured in a normal signal time interval when the movement value digitized by the motion determination unit is equal to or less than the predetermined threshold value and are already stored in the data storage unit, the electrocardiogram signal correction unit may correct the electrocardiogram waveform data in the abnormal signal time interval.

In the system for monitoring the heart condition in the companion animal according to an embodiment of the present disclosure, the electrocardiogram signal correction unit may include a learning model that is learned using the electrocardiogram waveform data, the phonocardiogram waveform data, and the ballistocardiogram waveform data measured and already stored in the normal signal time interval, receives the phonocardiogram waveform data and the ballistocardiogram waveform data measured in the abnormal signal time interval, and outputs the corrected electrocardiogram waveform data.

The system for monitoring the heart condition in the companion animal according to an embodiment of the present disclosure may further include a disease determination unit that determines the heart condition in the companion animal based on the cardiogram-integrated data.

The features and advantages of the present disclosure will become more apparent from the following detailed description based on the accompanying drawings.

Before describing the present disclosure, terms or words used in the present specification and claims should not be interpreted in usual and dictionary meanings, but should be interpreted in meanings and concepts that conform to the technical idea of the present disclosure based on the principle that the inventor can appropriately define the concept of the term in order to explain his or her own invention in the best way.

According to the present disclosure, since the electrocardiogram of a companion animal is measured through a non-contact capacitive electrocardiogram sensor, there is no need to bring electrodes into contact with the skin of the companion animal, so that there is no need for prior preparation such as hair removal and an electrocardiogram signal can be accurately collected without restricting the movement of the companion animal.

In addition, in addition to the electrocardiogram signal, a phonocardiogram signal and a ballistocardiogram signal collected and merged to generate cardiogram-integrated data, so that the heart condition of the companion animal can be ascertained more precisely.

### BRIEF DESCRIPTION OF THE DRAWINGS

FIG. 1 is a configuration diagram of a system for monitoring a heart condition in a companion animal according to an embodiment of the present disclosure.

FIG. 2 is a diagram illustrating an example of a block diagram of a capacitive electrocardiogram sensor illustrated in FIG. 1.

FIG. 3 is a diagram for explaining a state in which a sensor-integrated body illustrated in FIG. 1 is attached to a wearable member and worn on a heart area of a companion dog.

FIG. 4 is a diagram for explaining a state in which a phonocardiogram sensor illustrated in FIG. 1 is mounted.

FIG. 5 is a diagram for explaining the positions of electrodes of capacitive electrocardiogram sensors, a phonocardiogram sensor, and a ballistocardiogram sensor in a state in which a sensor-integrated body illustrated in FIG. 2 is attached to a wearable member and worn on a heart area of a companion dog.

FIG. 6 is a diagram for explaining cardiogram-integrated data in which electrocardiogram waveform data, phonocardiogram waveform data, and ballistocardiogram waveform data generated by a system for monitoring a heart condition in a companion animal according to an embodiment of the present disclosure are merged.

FIG. 7 is a configuration diagram of a system for monitoring a heart condition in a companion animal according to another embodiment of the present disclosure.

FIG. 8 is a diagram for explaining a process in which a system for monitoring a heart condition in a companion animal according to another embodiment of the present disclosure corrects an electrocardiogram signal.

### DETAILED DESCRIPTION

Terms used in the present specification are used for describing exemplary embodiments while not limiting the present disclosure. A singular form in the present specification may include a plural form unless specifically mentioned. The meaning of “comprise” and “comprising” used in the specification does not exclude the presence or addition of one or more other components in addition to the mentioned components. Throughout the specification, like reference numerals represent the same components, and the term “and/or” includes each of mentioned components and one or more combinations thereof. Although the terms “first” and “second” are used to describe various components, the components are not limited by the terms. The terms are used only to distinguish one element from another element. Therefore, a first component described below may be a second component within the technical idea of the present disclosure.

Throughout the specification, when a certain part is referred to as “including” a certain component, it indicates that the part might not exclude but further include other components, unless referred to the contrary. In addition, a term such as “ . . . unit” and “ . . . module” described in the specification means a unit for processing at least one function or operation, and this may be implemented with hardware, software, or a combination of the hardware and the software.

Hereinafter, various embodiments of the present disclosure are described with reference to the accompanying drawings.

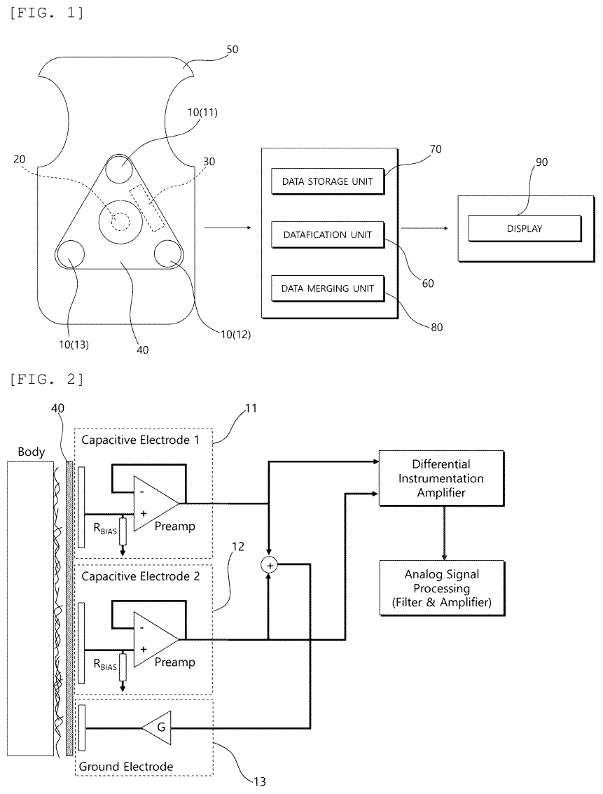

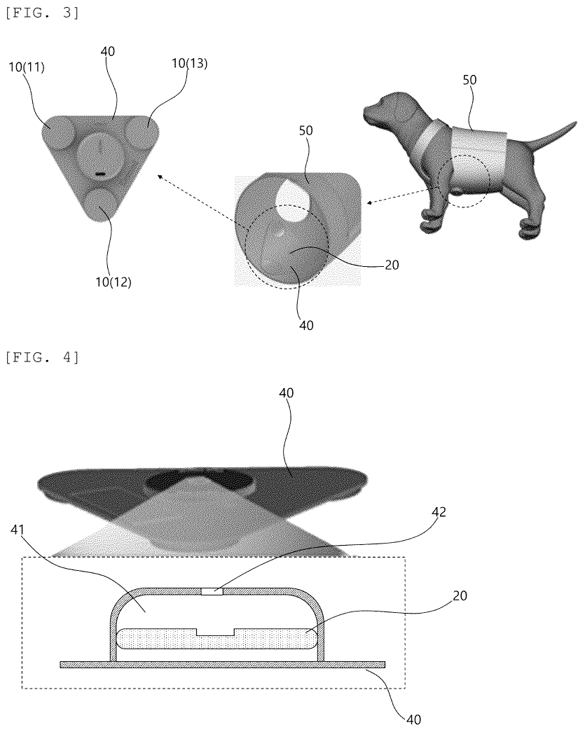

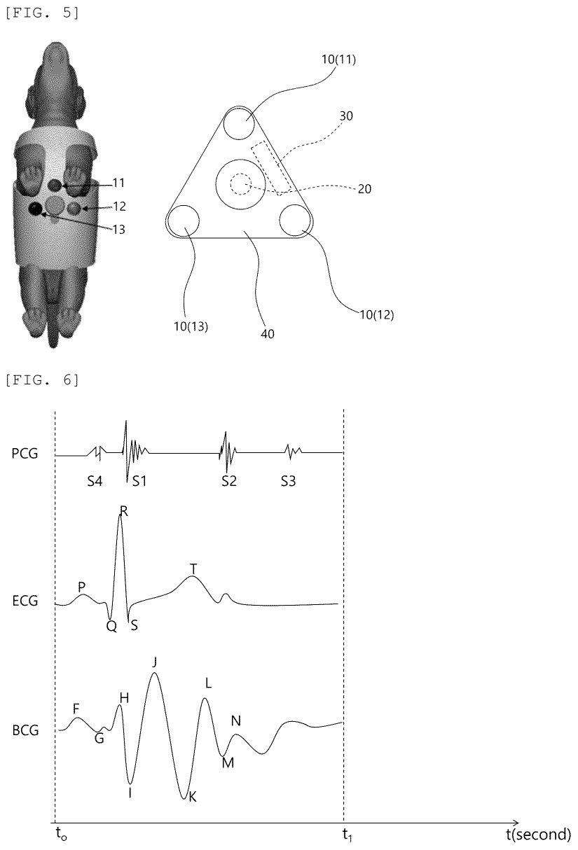

FIG. 1 is a configuration diagram of a system for monitoring a heart condition in a companion animal according to an embodiment of the present disclosure, and FIG. 2 is a diagram illustrating an example of a block diagram of a capacitive electrocardiogram sensor illustrated in FIG. 1. FIG. 3 is a diagram for explaining a state in which a sensor-integrated body illustrated in FIG. 1 is attached to a wearable member and worn on a heart area of a companion dog, and FIG. 4 is a diagram for explaining a state in which a phonocardiogram sensor illustrated in FIG. 1 is mounted. FIG. 5 is a diagram for explaining the positions of electrodes of capacitive electrocardiogram sensors, a phonocardiogram sensor, and a ballistocardiogram sensor in a state in which the sensor-integrated body illustrated in FIG. 2 is attached to the wearable member and worn on the heart area of the companion dog.

As illustrated in FIGS. 1 to 5, the system for monitoring a heart condition in a companion animal according to an embodiment of the present disclosure includes a capacitive electrocardiogram sensor 10 including a first electrode 11, a second electrode 12, and a third electrode 13 not in contact with the skin of a companion animal and collecting an electrocardiogram signal of the companion animal based on capacitive coupling, a phonocardiogram sensor 20 that collects a phonocardiogram signal of the companion animal, a ballistocardiogram sensor 30 that collects a ballistocardiogram signal of the companion animal, a sensor-integrated body 40 in which the capacitive electrocardiogram sensor 10, the phonocardiogram sensor 20, and the ballistocardiogram sensor 30 are arranged, a wearable member 50 that is attached with the sensor-integrated body 40 and is worn by the companion animal, the sensor-integrated body 40 being arranged toward a heart area of the companion animal, a datafication unit 60 that generates electrocardiogram waveform data, phonocardiogram waveform data, and ballistocardiogram waveform data by datafying the collected electrocardiogram signal, phonocardiogram signal, and ballistocardiogram signal over time, a data storage unit 70 that stores the generated electrocardiogram waveform data, phonocardiogram waveform data, and ballistocardiogram waveform data, a data merging unit 80 that generates cardiogram-integrated data by merging the generated electrocardiogram waveform data, phonocardiogram waveform data, and ballistocardiogram waveform data, and a display 90 that displays the cardiogram-integrated data.

The present disclosure relates to a system for monitoring a heart condition in a companion animal, and can monitor the heart condition by integrating the cardiograms of the companion animal through a non-restrained wearable device that integrates electrocardiogram, ballistocardiogram, and phonocardiogram generated according to the heart motion of the companion animal. In the related art, since electrocardiogram measurement for diagnosing a heart condition in a companion animal requires contact of electrodes with the skin of the companion animal, prior preparation such as hair removal for exposing the skin is required, and the movement of the companion animal needs to be restricted so that the electrodes do not fall off, making it difficult to accurately measure the electrocardiogram. As a solution to this problem, the present disclosure has been devised.

Specifically, the system for monitoring a heart condition in a companion animal according to an embodiment of the present disclosure includes the capacitive electrocardiogram sensor 10, the phonocardiogram sensor 20, the ballistocardiogram sensor 30, the sensor-integrated body 40, the wearable member 50, the datafication unit 60, the data storage unit 70, the data merging unit 80, and the display 90.

The capacitive electrocardiogram sensor 10 is a sensor that collects the electrocardiogram signal of the companion animal based on capacitive coupling. The electrocardiogram (ECG) represents the electrical changes of the heart from the sinus node to the contraction of the left ventricle, and includes five waveforms: P wave, Q wave, R wave, S wave, and T wave (see FIG. 6). In the case of a normal companion animal, the electrocardiogram waveform is repeated periodically, but when there is a heart condition, since the heart motion is irregular, the periodicity of the electrocardiogram waveform may be irregularly deformed. The capacitive coupling is a phenomenon in which energy is transferred from one circuit to the other circuit due to the mutual capacitance between the two circuits separated from each other. That is, the capacitive coupling is a phenomenon in which the energy of an AC signal source is transferred to the other circuit through a dielectric or air.

The capacitive electrocardiogram sensor 10 includes the first electrode 11, the second electrode 12, and the third electrode 13. The first electrode 11, the second electrode 12, and the third electrode 13 are based on the capacitive coupling, and thus do not come into direct contact with the skin of the companion animal. The capacitive electrocardiogram sensor 10 is arranged in the sensor-integrated body 40, and the first electrode 11, the second electrode 12, and the third electrode 13 may be arranged on the surface of the sensor-integrated body 40 or built into the interior thereof. The first electrode 11, the second electrode 12, and the third electrode 13 may not come into direct contact with the skin of the companion animal through the sensor-integrated body 40 or the air. In such a case, among the areas of the sensor-integrated body 40, at least an area located between the first electrode 11, the second electrode 12, and the third electrode 13 and the skin of the companion animal may be made of an insulating material.

The first electrode 11, the second electrode 12, and the third electrode 13 can correspond to an N pole, a P pole, and a G pole, respectively. The first electrode 11 corresponding to the N pole is a cathode serving as a reference point, and can be a relative reference compared to the P pole for the electrical flow of the heart and form an electrocardiogram signal by forming a pair with the P pole. The second electrode 12 is the P pole (anode) of the electrocardiogram signal, and can be located at a site where the electrical activity of the heart is strongly detected. The third electrode 13 is a ground or a reference electrode (G pole) and is located at a site of the companion animal's body where an electrical signal change is relatively small.

Referring to FIG. 2, the capacitive electrodes of the capacitive electrocardiogram sensor 10, that is, the first electrode 11 and the second electrode 12 are implemented with linear amplifiers having an infinite input impedance R_{BIAS}, and can amplify minute and weak electrocardiogram signals. In addition, the capacitive electrocardiogram sensor 10 can be implemented with a differential measuring amplifier, an analog signal processing unit, or the like in addition to the capacitive electrodes, and can collect electrocardiogram signals. However, since the block diagram of the capacitive electrocardiogram sensor 10 illustrated in FIG. 2 is merely an example, the capacitive electrocardiogram sensor 10 is not necessarily limited thereto.

The phonocardiogram sensor 20 is a sensor that measures the phonocardiogram signal of the companion animal. The phonocardiogram (PCG) is a sound produced by a beating heart and the resulting blood flow, and is classified into a first heart sound S1, a second heart sound S2, a third heart sound S3, and a fourth heart sound S4 (see FIG. 6). The first heart sound S1 is a sound produced when the atrioventricular valve closes, and the second heart sound S2 is a sound produced when the aortic and pulmonary valves close. The first heart sound S1 to the fourth heart sound S4 are repeated periodically, but when there is a heart condition, since the periodicity may be irregular or the sound may be different, the heart condition can be diagnosed using phonocardiography. However, it is not easy to distinguish the first heart sound S1 from the second heart sound S2 by auscultation alone. Therefore, it is not almost possible for even veterinarians to distinguish the period by auscultation alone and diagnose a heart condition based on the period.

The phonocardiography sensor 20 can use a MEMS microphone, but is not necessarily limited thereto and various acoustic sensors can be used.

The ballistocardiogram sensor 30 is a sensor that collects the ballistocardiogram signal of the companion animal. The ballistocardiogram (BCG) is a micromovement or vibration resulting from a heartbeat, and its signal is classified into F wave, G wave, H wave, I wave, J wave, K wave, L wave, M wave, N wave, and the like (see FIG. 6). The ballistocardiogram signal also shows periodicity of the waveform within the normal state data of the companion animal, but when the companion animal has a heart condition, the periodicity or waveform may be abnormally deformed.

The ballistocardiogram sensor 30 may use a pressure sensor or a piezoelectric sensor. For example, a PVDF sensor may be used, but the ballistocardiogram sensor 30 is not necessarily limited thereto.

The sensor-integrated body 40 is a member in which the capacitive electrocardiogram sensor 10, the phonocardiogram sensor 20, and the ballistocardiogram sensor 30 are integrated and arranged. The phonocardiogram sensor 20 may be arranged at the center of the sensor-integrated body 40. One first electrode 11, one second electrode 12, and one third electrode 13 of the capacitive electrocardiogram sensor 10 may be arranged at vertices of an imaginary triangle centered on the phonocardiogram sensor 20, respectively. The triangular structure in which the first electrode 11, the second electrode 12, and the third electrode 13 are arranged in this way corresponds to the Einthoven's triangle suitable for electrocardiogram measurement. The ballistocardiogram sensor 30 may be arranged between two of the first electrode 11, the second electrode 12, and the third electrode 13. The surface of the sensor-integrated body 40 surrounds the capacitive electrocardiogram sensor 10, the phonocardiogram sensor 20, and the ballistocardiogram sensor 30, so that the sensors may be built into the interior of the sensor-integrated body 40. However, all the capacitive electrocardiogram sensor 10, the phonocardiogram sensor 20, and the ballistocardiogram sensor 30 do not necessarily have to be built into the interior of the sensor-integrated body 40, and all or a part thereof may be exposed on an outer surface of the sensor-integrated body 40.

The wearable member 50 is a clothing member worn by the companion animal. Such a wearable member 50 may be formed in the form of a strap, a top, a vest, or a combination thereof, and there is no particular limitation in its shape or material as long as it can be worn by the companion animal while covering the heart area of the companion animal. The sensor-integrated body 40 is attached to an area of the inner surface of the wearable member 50 that faces the heart area of the companion animal. Accordingly, the sensor-integrated body 40 is located toward the heart area of the companion animal. One surface of the sensor-integrated body 40 faces the heart area of the companion animal, and the other surface of the sensor-integrated body 40 faces the inner surface of the wearable member 50 worn by the companion animal (see FIG. 3).

In order to effectively measure the heart sounds, the phonocardiogram sensor 20 may be arranged on one surface of the sensor-integrated body 40 that faces the heart area of the companion animal. An internal space 41 in which the phonocardiogram sensor 20 is embedded may be provided on one surface of the sensor-integrated body 40. In addition, a through hole 42, through which the internal space 41 and the heart area communicate with each other, may be provided so that the heart sounds can be introduced into the internal space 41 (see FIG. 4).

The positions of the first electrode 11, the second electrode 12, and the third electrode 13 are determined according to the position of the sensor-integrated body 40. The sensor-integrated body 40 can be attached to the wearable member 50 so that the first electrode 11 serving as the N pole is arranged to face between two front legs of the companion animal and the second electrode 12 serving as the P pole is arranged to face an area between the back of the front legs closest to the heart and the lungs. The ballistocardiogram sensor 30 may be arranged between the first electrode 11 and the second electrode 12 (see FIG. 5).

The datafication unit 60, the data storage unit 70, and the data merging unit 80 process the electrocardiogram signal, the phonocardiogram signal, and the ballistocardiogram signal collected from the capacitive electrocardiogram sensor 10, the phonocardiogram sensor 20, and the ballistocardiogram sensor 30, and all or a part thereof may be built into the sensor-integrated body 40 or may be included in an external device separate from the sensor-integrated body 40. The external device refers to any electronic device that processes and stores data, such as a server, a system, a mobile terminal, or a computer. The mobile terminal may include any type of handheld-based wireless communication device that can be connected to a web server through a network, such as a mobile phone, a smart phone, a personal digital assistant (PDA), a portable multimedia player (PMP), or a tablet PC. The sensor-integrated body 40 may include a communication module (not illustrated) that can transmit the electrocardiogram signal, the phonocardiogram signal, and the ballistocardiogram signal to the external device. A communication network used by the communication module to perform communication can be configured regardless of a communication mode such as wired or wireless communication, and can be implemented as various communication networks such as a local area network (LAN), a metropolitan area network (MAN), and a wide area network (WAN). According to an embodiment, the communication module can perform communication with the external device or the like by utilizing a low-power Bluetooth (BLE: Bluetooth Low Energy) technology, and alternatively, it can utilize a wireless fidelity (WiFi) technology.

The datafication unit 60 can generate the electrocardiogram waveform data, the phonocardiogram waveform data, and the ballistocardiogram waveform data by datafying the collected electrocardiogram signal, phonocardiogram signal, and ballistocardiogram signal over time.

The data storage unit 70 stores the electrocardiogram waveform data, the phonocardiogram waveform data, and the ballistocardiogram waveform data generated by the datafication unit 60. The data storage unit 70 may include a memory, a cache, a buffer, or the like, and may be configured by software, firmware, hardware, or a combination of at least two of these. According to an embodiment, the data storage unit 70 may be configured in a form such as a micro SD card.

The data merging unit 80 generates the cardiogram-integrated data by merging the electrocardiogram waveform data, the phonocardiogram waveform data, and the ballistocardiogram waveform data generated by the datafication unit 60. The electrocardiogram waveform data, the phonocardiogram waveform data, and the ballistocardiogram waveform data may be generated in the form of graphs for an electrocardiogram waveform, a phonocardiogram waveform, and a ballistocardiogram waveform.

Since the electrocardiogram waveform data, the phonocardiogram waveform data, and the ballistocardiogram waveform data are obtained by datafying the electrocardiogram signal, the phonocardiogram signal, and the ballistocardiogram signal over time, they can be synchronized and merged over time. The cardiogram-integrated data merged in this way can be visualized as one graph for the electrocardiogram waveform, the phonocardiogram waveform, and the ballistocardiogram waveform (see FIG. 6). The phonocardiogram waveform appearing after the R-spike of the electrocardiogram waveform can be recognized as the first heart sound S1 and the second heart sound S2 in order, so that the first heart sound S1 and the second heart sound S2, which are not easily distinguished by auscultation alone, can be easily distinguished. In addition, since the relationship among the electrocardiogram waveform, the phonocardiogram waveform, and the ballistocardiogram waveform over time is shown in an integrated manner, a heart condition can be diagnosed early based on the graph.

The display 90 displays the cardiogram-integrated data generated by the data merging unit 80. The display 90 may be included in the above-described external device together with all or a part of the datafication unit 60, the data storage unit 70, and the data merging unit 80, or may be included in a separate device. For example, the display 90 may be a separate output device managed by a veterinarian.

As shown in Table 1 below, a heart condition in the companion animal can be diagnosed through the electrocardiogram, the phonocardiogram, and the ballistocardiogram, so that the heart condition can be diagnosed early and accurately based on the necessary waveforms in the cardiogram-integrated data.

TABLE 1

| Type | Heart condition | Electrocardiogram | Phonocardiogram | Ballistocardiogram |
|---|---|---|---|---|
| Acquired | Heart failure | ◯ | | ◯ |
| heart condition | Dilated | ◯ | | ◯ |
| | cardiomyopathy | | | |
| | Mitral valve | ◯ | ◯ | |
| | insufficiency | | | |
| | Aortic regurgitation | ◯ | ◯ | |
| | Tricuspid stenosis | ◯ | ◯ | |
| Congenital | Patent ductus | ◯ | ◯ | |
| heart condition | arteriosus | | | |
| | Pulmonary stenosis | ◯ | ◯ | |
| | Aortic stenosis | ◯ | ◯ | |
| | Ventricular septal | ◯ | | |
| | defect | | | |
| Acquired/congenital | Coronary artery | ◯ | ◯ | ◯ |
| heart condition | disease | | | |

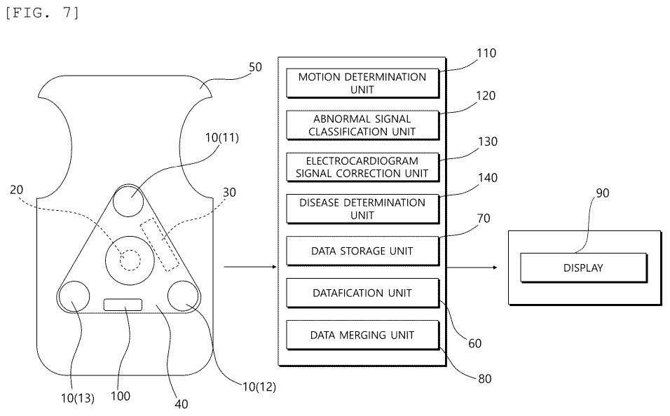

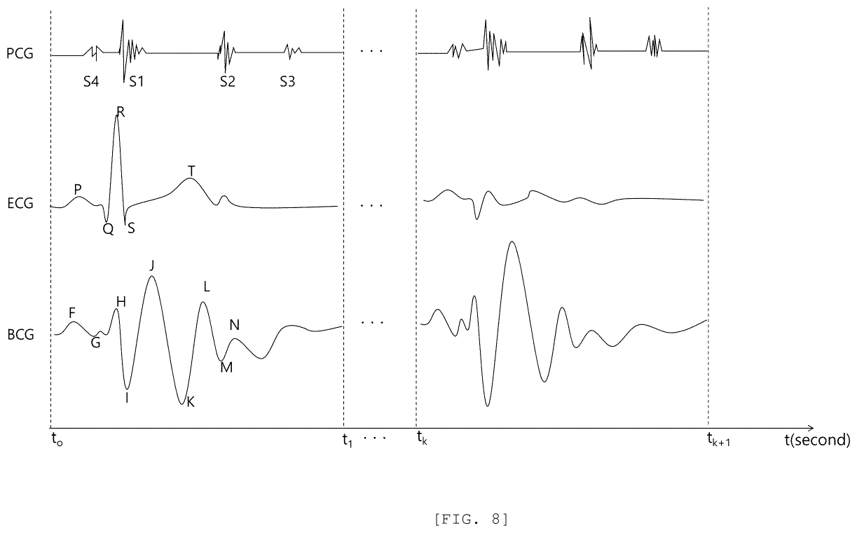

FIG. 7 is a configuration diagram of a system for monitoring a heart condition in a companion animal according to another embodiment of the present disclosure, and FIG. 8 is a diagram for explaining a process in which the system for monitoring a heart condition in a companion animal according to another embodiment of the present disclosure corrects an electrocardiogram signal.

Referring to FIG. 7, the system for monitoring a heart condition in a companion animal according to another embodiment of the present disclosure may further include an acceleration sensor 100, a motion determination unit 110, an abnormal signal classification unit 120, and an electrocardiogram signal correction unit 130.

The aforementioned capacitive electrocardiogram sensor 10 can collect the electrocardiogram signal without contacting the skin of the companion animal, but when the companion animal moves violently, the first electrode 11, the second electrode 12, and the third electrode 13 may be deviated from their original positions and thus an abnormal signal in which the electrocardiogram signal is distorted or lost may be collected. Accordingly, the system for monitoring a heart condition in a companion animal according to another embodiment of the present disclosure further includes the acceleration sensor 100, the motion determination unit 110, the abnormal signal classification unit 120, and the electrocardiogram signal correction unit 130 as means for detecting the collection of the abnormal signal and correcting the electrocardiogram signal.

The acceleration sensor 100 is mounted on the sensor-integrated body 40 and measures acceleration. Since the acceleration sensor 100 is mounted on the sensor-integrated body 40, it can detect whether the companion animal is moving, the movement speed thereof, or the like. Such an acceleration sensor 100 can measure acceleration in the X, Y, and Z-axis directions.

The motion determination unit 110 digitizes the movement of the companion animal based on the acceleration measured by the acceleration sensor 100. The numerical value of the movement can be expressed as an acceleration value in each of the X, Y, and Z-axis directions. The digitized movement value can be synchronized with the time at which the electrocardiogram sensor 10, the phonocardiogram sensor 20, and the ballistocardiogram sensor 30 collect respective signals, and can be datafied by time. When the digitized movement value exceeds a preset predetermined threshold value, the motion determination unit 110 determines that the companion animal is moving abnormally, and records the time interval during which the threshold value is exceeded. The time interval determined as the abnormal movement may correspond to an abnormal signal time interval (see “tₖ to tₖ₊₁” interval in FIG. 8) in which the electrocardiogram signal is distorted or lost. When the digitized movement value is equal to or less than the preset threshold value, the motion determination unit 110 can record the interval as a normal signal time interval (see “tₒ to t₁” interval in FIG. 8). Only electrocardiogram waveform data, phonocardiogram waveform data, and ballistocardiogram waveform data collected and generated in the normal signal time interval can be selectively stored in the data storage unit 70.

The abnormal signal classification unit 120 classifies, as an abnormal signal, an electrocardiogram signal measured in the abnormal signal time interval.

The electrocardiogram signal correction unit 130 corrects electrocardiogram waveform data in the abnormal signal time interval. Based on the electrocardiogram waveform data, the phonocardiogram waveform data, and the ballistocardiogram waveform data collected and generated in the normal signal time interval and stored in the data storage unit 70, the electrocardiogram signal correction unit 130 can correct the electrocardiogram waveform data in the abnormal signal time interval.

For example, an I-J waveform can be extracted from the ballistocardiogram waveform data so as to correspond to the time corresponding to a portion of the electrocardiogram waveform data where data is lost or distorted, and the portion of the electrocardiogram waveform data where data is lost or distorted can be corrected based on the extracted I-J waveform. The I-J waveform showing the largest amplitude in the ballistocardiogram waveform can be extracted from the ballistocardiogram waveform data, and the portion of the electrocardiogram waveform data where data is lost or distorted can be generated and supplemented based on the amplitude, shape, or the like of the I-J waveform. In such a case, a J waveform (J-peak) captured on the ballistocardiogram waveform data can be shifted to the time axis on the ballistocardiogram waveform graph to generate an R waveform (R-spike) in the lost or distorted portion of the electrocardiogram waveform data and correct the portion.

As another example, the electrocardiogram signal correction unit 130 can correct the electrocardiogram waveform data by using a learning model. The learning model is a machine learning model trained to perform a task by using an algorithm or a series of predefined steps. Such a learning model can be learned using, as a learning data set, the electrocardiogram waveform data, the phonocardiogram waveform data, and the ballistocardiogram waveform data measured and already stored in the normal signal time interval. The learning data set can be configured as a data set in which a correct answer (label) exists, and the electrocardiogram waveform data can be utilized as label data. Accordingly, the learning model can receive the phonocardiogram waveform data and the ballistocardiogram waveform data measured in the abnormal signal time interval, and output the corrected electrocardiogram waveform data.

The system for monitoring heart conditions in a companion animal according to another embodiment of the present disclosure may further include a disease determination unit 140.

The disease determination unit 140 can determine the heart condition in the companion animal based on the cardiogram-integrated data. Referring to Table 1 above, the electrocardiogram waveform data, the ballistocardiogram waveform data, and the phonocardiogram waveform data have unique waveform shapes or irregularities for each heart condition. Accordingly, by setting in advance the electrocardiogram waveform data, the ballistocardiogram waveform data, and the phonocardiogram waveform data that can distinguish the heart condition and inputting the cardiogram-integrated data generated in real time, the heart condition in the companion animal can be diagnosed early.

Although the present disclosure has been described in detail through specific embodiments and experimental examples, this is for specifically explaining the present disclosure, the present disclosure is not limited thereto, and it is clear that modifications or improvements can be made by those skilled in the art within the technical concept of the present disclosure. All simple modifications or changes of the present disclosure fall within the scope of the present disclosure, and the specific scope of protection of the present disclosure will be clarified by the appended claims.

What is claimed is:

1. A system for monitoring a heart condition in a companion animal, the system comprising:
a capacitive electrocardiogram sensor including a first electrode, a second electrode, and a third electrode not in contact with a skin of a companion animal and collecting an electrocardiogram signal of the companion animal based on capacitive coupling;
a phonocardiogram sensor that collects a phonocardiogram signal of the companion animal;
a ballistocardiogram sensor that collects a ballistocardiogram signal of the companion animal;
a sensor-integrated body in which the capacitive electrocardiogram sensor, the phonocardiogram sensor, and the ballistocardiogram sensor are arranged;
a wearable member that is attached with the sensor-integrated body and is worn by the companion animal, the sensor-integrated body being arranged toward a heart area of the companion animal;
a datafication unit that generates electrocardiogram waveform data, phonocardiogram waveform data, and ballistocardiogram waveform data by datafying the collected electrocardiogram signal, phonocardiogram signal, and ballistocardiogram signal over time;
a data storage unit that stores the generated electrocardiogram waveform data, phonocardiogram waveform data, and ballistocardiogram waveform data;
a data merging unit that generates cardiogram-integrated data by merging the generated electrocardiogram waveform data, phonocardiogram waveform data, and ballistocardiogram waveform data; and
a display that displays the cardiogram-integrated data.

2. The system of claim 1, wherein:
in the sensor-integrated body, the phonocardiogram sensor is arranged at a center of the sensor-integrated body,
one first electrode, one second electrode, and one third electrode are arranged at vertices of an imaginary triangle centered on the phonocardiogram sensor, respectively, and
the ballistocardiogram sensor is arranged between two of the first electrode, the second electrode, and the third electrode.

3. The system of claim 1, wherein one surface of the sensor-integrated body that faces a skin of the companion animal is provided with an internal space in which the phonocardiogram sensor is embedded and a through hole through which heart sounds are introduced into the internal space.

4. The system of claim 3, wherein the first electrode is arranged between two front legs of the companion animal.

5. The system of claim 1, further comprising:
an acceleration sensor mounted on the sensor-integrated body and measuring acceleration;
a motion determination unit that digitizes a movement of the companion animal based on the acceleration measured by the acceleration sensor;
an abnormal signal classification unit that classifies, as an abnormal signal, the electrocardiogram signal measured in an abnormal signal time interval in which a movement value digitized by the motion determination unit exceeds a predetermined threshold value; and
an electrocardiogram signal correction unit that corrects the electrocardiogram waveform data in the abnormal signal time interval.

6. The system of claim 5, wherein, based on the electrocardiogram waveform data, the phonocardiogram waveform data, and the ballistocardiogram waveform data that are measured in a normal signal time interval when the movement value digitized by the motion determination unit is equal to or less than the predetermined threshold value and are already stored in the data storage unit, the electrocardiogram signal correction unit corrects the electrocardiogram waveform data in the abnormal signal time interval.

7. The system of claim 6, wherein the electrocardiogram signal correction unit comprises:
a learning model that is learned using the electrocardiogram waveform data, the phonocardiogram waveform data, and the ballistocardiogram waveform data measured and already stored in the normal signal time interval, receives the phonocardiogram waveform data and the ballistocardiogram waveform data measured in the abnormal signal time interval, and outputs the corrected electrocardiogram waveform data.

8. The system of claim 1, further comprising:
a disease determination unit that determines the heart condition in the companion animal based on the cardiogram-integrated data.
